(19) United States
(12) Patent Application Publication
(10) Pub. No.: US 2024/0038344 A1
(43) Pub. Date: Feb. 1, 2024

(54) ORCHESTRATOR ENGINE TO PROVIDE CONTEXT DRIVEN WORKFLOW OF RADIOLOGY IMAGES AND PATIENT DATA TO ARTIFICIAL INTELLIGENCE ENGINES FOR FURTHER PROCESSING

(71) Applicant: RADIOLOGY PARTNERS, INC., El Segundo, CA (US)

(72) Inventors: Nina KOTTLER, San Diego, CA (US); Thomas N. Tobias, Boulder, CO (US)

(21) Appl. No.: 18/362,924

(22) Filed: Jul. 31, 2023

Related U.S. Application Data
(60) Provisional application No. 63/370,036, filed on Aug. 1, 2022.

(51) Int. Cl. G16H 10/60

(52) U.S. Cl. CPC G16H 10/60

(57) ABSTRACT

Provided are a computer program product, system, and method for an orchestrator engine to provide context driven workflow of radiology images and patient data to artificial intelligence engines for further processing. An orchestrator engine, providing context driven workflow, processes the patient information to determine an artificial intelligence program of a plurality of artificial intelligence programs to process the medical image. The artificial intelligence program processes the medical image and the patient data to provide a structured result as output. The structured result with the patient information is forwarded to the radiologist to evaluate the medical image.

### CROSS-REFERENCE TO RELATED APPLICATION

This application claims the benefit of U.S. Provisional Application No. 63/370,036, filed Aug. 1, 2022, which provisional application is incorporated herein by reference in its entirety.

### STATEMENT REGARDING PRIOR DISCLOSURES BY THE INVENTOR OR A JOINT INVENTOR UNDER 37 C.F.R 1.77(B)(6)

The following Disclosure is submitted under 35 U.S.C. 102(b)(1)(A): On Aug. 1, 2021, inventor Nina Kottler, M.D., made a presentation at the University of California at San Diego (UCSD) titled “The Future of AI in Radiology”. A video is recorded of this presentation and available on a UCSD web page at https://radiology.ucsd.edu/about/alumni/events.html. A transcript of this video, generated by YouTube®, and a copy of slides shown during this presentation, and shown in the video, will be included in an Information Disclosure Statement filed with the present Application. (YouTube is a registered trademark throughout the world of Google LLC).

### BACKGROUND OF THE INVENTION

### 1. Field of the Invention

The present invention relates to a computer program product, system, and method for an orchestrator engine to provide context driven workflow of radiology images and patient data to artificial intelligence engines for further processing.

### 2. Description of the Related Art

In the 2000s, radiologists used the PACS system, Picture Archiving and Communication System, which provides storage and access to images from different source machines. As the workload of radiologists increased, PACS helped radiologist manage the increased workload. From 2000-2011, the volume of radiology work from Medicare increased by about 80% even though the number of radiologists increased by only about 25%. PACS systems helped radiologists keep up with the increased workload. However, PACS separated the radiologists from the clinician and the benefit of receiving insight and input from the clinician. This lack of input from the physician is an unintended drawback of PACS.

Some of the factors preventing the widespread adoption of artificial intelligence (AI) are hype over the maturity of AI, the lack of a strong business case, the difficulty in accessing and normalizing medical data, inherent data and hence AI model bias, AI's lack of contextual understanding of data without specific training, the difficulty of data orchestration, and difficulty in deployment of AI technology within the complex medical environment. With respect to the maturity of modern AI techniques, like most new technology, the impact is overestimated in the short term and underestimated in the long run. Currently, we are at a point of supplier consolidation as disillusionment that the high expectations of AI for healthcare have not fully panned out. FIG. 1 is a Gartner hype curve showing how new technology is adopted over time, with the x-axis showing time and the y-axis visibility. With respect to healthcare, the current location in the Gartner curve for AI adoption is between inflated expectations and a trough of disillusionment, such as from negative press, which results in supplier consolidation as shown in FIG. 2, which is occurring now with AI technology for healthcare.

Currently, there are not enough radiologists to keep up with the increased demand for radiology services. This means there is a business case for deploying AI with radiology to improve the efficiency of radiologists to increase their output. In this way, the business case favors the adoption of AI.

The deployment of AI in radiology services requires huge amounts of data to train the AI programs. One problem is radiology data tends to be unstructured, but structured data is required to train the AI programs to analyze images and data to provide diagnosis and report data. Further, radiologists do not have time to label data to provide structure. Yet further, different PACS companies store their data in proprietary formats that are not normalized. Even different radiology practices implementations of radiology data standards, such as DICOM (Digital Imaging and Communications in Medicine) and HL7 (Health Level 7) are maintained in non-standard formats across radiology groups and hospitals. This variability prevents the sharing of data to use for training the AI.

### SUMMARY

Provided are a computer program product, system, and method for an orchestrator engine to provide context driven workflow of radiology images and patient data to artificial intelligence engines for further processing. An orchestrator engine, providing context driven workflow, processes the patient information to determine an artificial intelligence program of a plurality of artificial intelligence programs to process the medical image. The artificial intelligence program processes the medical image and the patient data to provide a structured result as output. The structured result with the patient information is forwarded to the radiologist to evaluate the medical image.

### BRIEF DESCRIPTION OF THE DRAWINGS

FIGS. 1 and 2 illustrate a Gartner hype curve as known in the prior art.

FIGS. 3 and 4 provide examples of how training data may differ from the radiology practice.

FIG. 5 provides an example of differences in training data and radiologist data.

FIG. 6 illustrates an example of a life cycle of a radiology exam.

FIG. 7 illustrates an example of different algorithms that may be applied during the life cycle of the exam.

FIG. 8 illustrates an example of a user interface of patient information.

FIG. 9 illustrates an example of how patient information from the user interface of FIG. 8 is inserted into a patient report.

FIGS. 10 and 11 illustrate examples of output from a CAD (computer aided detection) triage (CADt) model.

FIGS. 12-22, 24 illustrate embodiments of orchestration of patient information to AI processing.

FIG. 23 illustrates an embodiment of the flow of operations.

FIGS. 25A, 25B, and 26 illustrate embodiments of operations performed by the orchestrator to select an artificial intelligence program and the selected artificial intelligence program to process the image and patient information.

FIG. 27 illustrates a computing environment in which embodiments may be implemented.

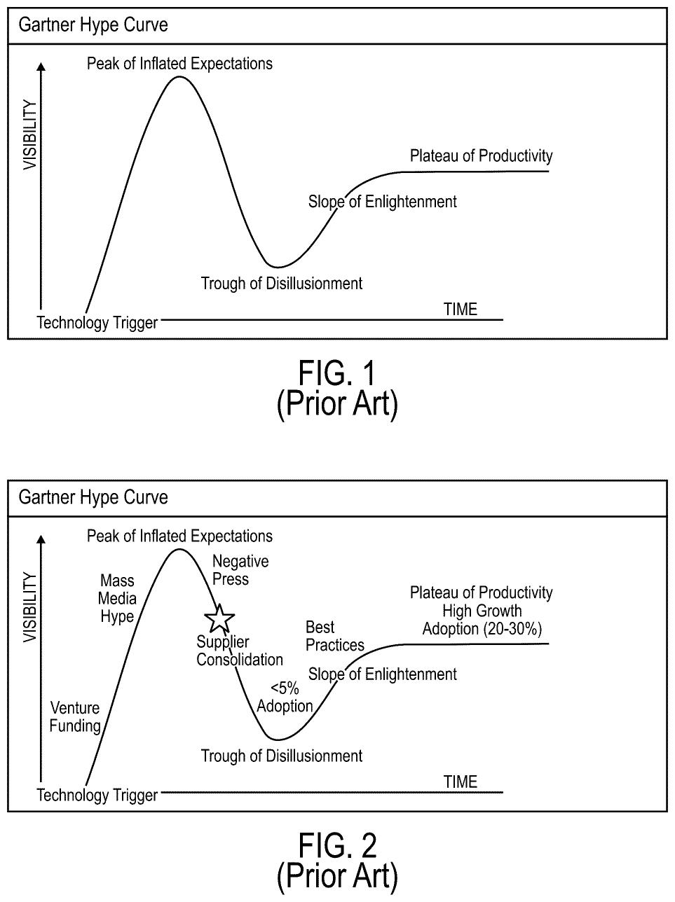

### DETAILED DESCRIPTION

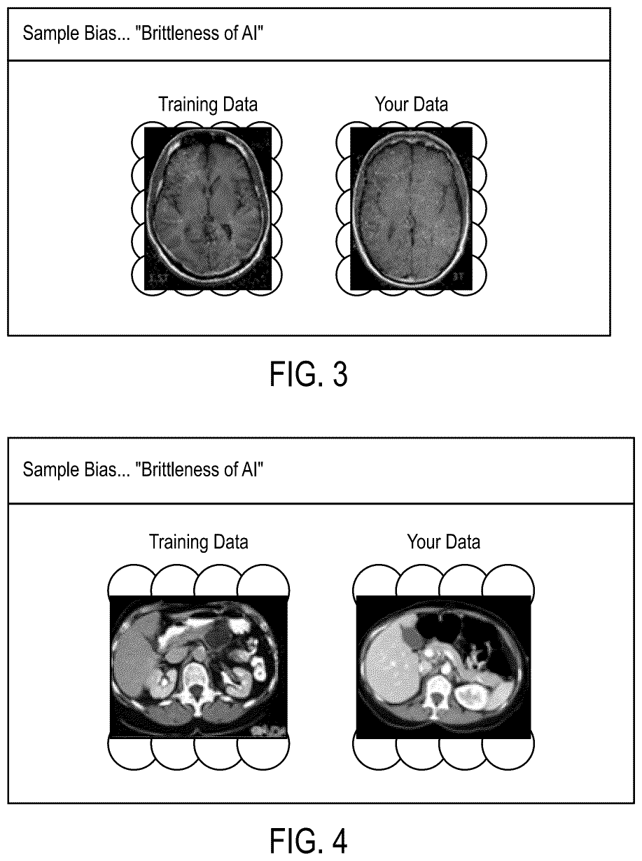

One issue with developing AI for radiology services is the bias of radiology data, which results in the brittleness of AI. The AI is trained on a certain set of data and produces a model based on the data on which it is trained. Then the model output from the AI applied to data is validated and the AI is further trained on the validated data. However, if a particular radiologist practice uses different data, such as with a different format, produced by different machines, etc., then the level of accuracy of the AI trained on different data may not be as high as expected. For instance, patient data may be biased if it uses different equipment to create the images, such as if the source machine being used has a different magnet strength than used to produce the data used to train the AI. FIGS. 3 and 4 show how the training data may differ from the radiology practice data (labeled “Your data”) where the AI is deployed due to differences in equipment that produce different images for the same patient. Performing different methodologies for studies may also result in different imaging data. For instance, the late arterial phase is the best for cancer detection, but portal venous phase is used for general pathology detection. The phase of contrast opacification used in training can affect the accuracy of the AI model results.

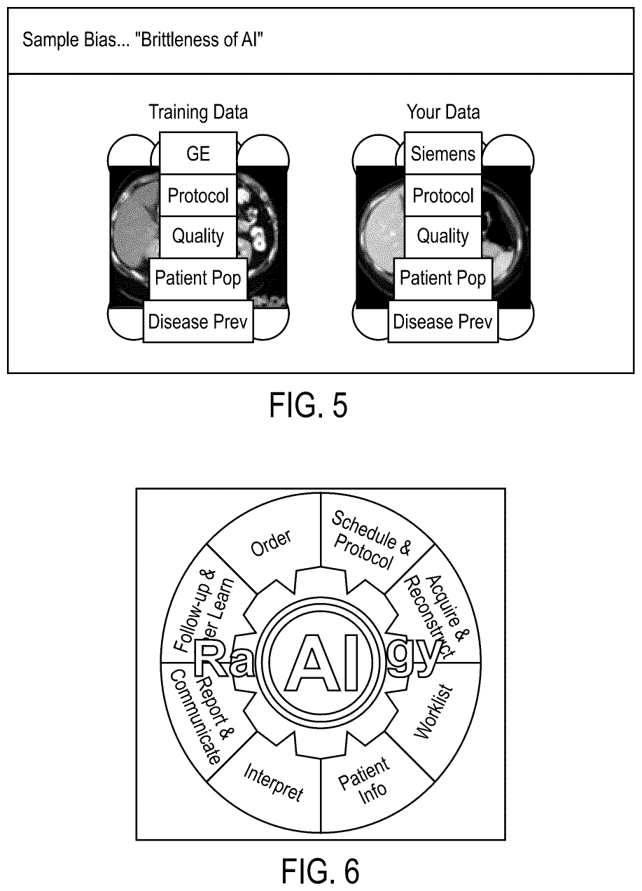

FIG. 5 shows the type of differences in training data and radiologist data (“Your Data) that may result in less accurate AI results when applied to the radiologist data. Differences in training data used to train the AI and the radiologist data where the AI is deployed may result from differences in: software used, scanner types, versions of the scanner, different protocols, patient population differences, disease prevalence in populations. For these reasons, the radiologists cannot simply trust the accuracy of the AI results when applied to their specific data. Further, many AI systems that produce good results in a lab may not translate to a real world clinical implementation, in part because the real world clinical data differs from the data on which the AI model was trained. Thus, AI tested in a closed laboratory environment, may not be as accurate when applied in the real world with different data unique to the radiologist practice in which it is deployed.

FIG. 6 shows the life cycle of a radiology exam beginning with a referring clinician having a clinical question in mind and ordering a study to determine the problem. A radiology examination is scheduled, and the images are acquired and reconstructed. At that point, the images are added to a worklist with information on the patient which is provided to the radiologist to interpret and diagnose. The radiologist adds context to a report and communicates those findings to the referring physician. The radiologist may then follow-up and peer learning on the exams is performed to ensure that patients are receiving appropriate care. AI may be inserted into each step in the process of FIG. 6.

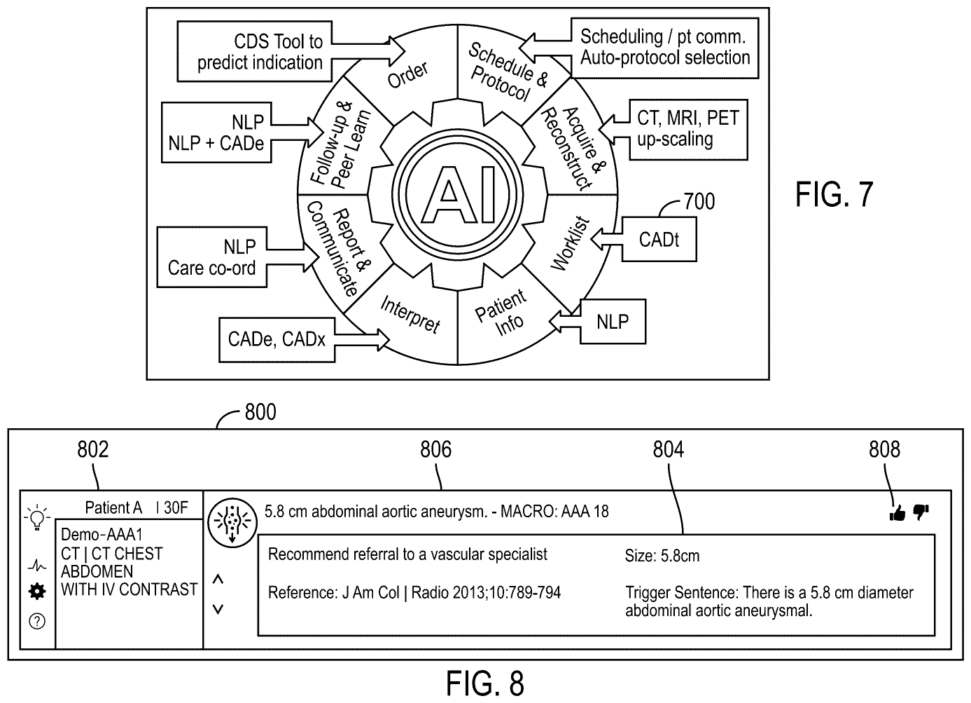

FIG. 7 shows the different algorithms that may be applied during the life cycle of the exam. For instance, AI models can be used to gather relevant patient information and provide that information to the radiologist for interpretation. AI models can also be used to reconstruct raw data so that low quality (low radiation dose or rapidly acquired images) data can be de-noised and presented to the radiologist at a quality level appropriate for interpretation. There are numerous Federal Drug Administration (FDA) cleared and approved algorithms that may be used for these processes.

Natural Language Processing (NLP) AI may be deployed at different phases in FIG. 7 to interpret the radiologist language and portions of a radiologist report to interpret the radiologist information in the report. For instance, the history of the radiology report may indicate a history of medical conditions, such as Cholangiocarcinoma and then the findings may say something totally different. In such a case, the NLP can determine the difference between those different segments of the report through parsing and interpreting the content.

There is considerable variability in radiology examination reports even though there are guidelines for different detected conditions, such as an Abdominal aortic aneurysm (AAA). Radiologists do not consistently apply the guidelines.

One program, recoMD® from Radiology Partners, trains the AI to parse and interpret radiology reports. The program is also trained with the information from medical journal articles and consensus papers so it can extract and structure relevant information from the report and apply that information to the programmed guidelines to produce the relevant recommendation as a clinical aid to the radiologist. The recoMD® program review what the radiologist is dictating about the exam and the patient, and extract the relevant information and use the extracted and interpreted language to lookup the best practice to provide information on the best practice and billing conditions. (recoMD is a registered trademark of Radiology Partners, Inc.).

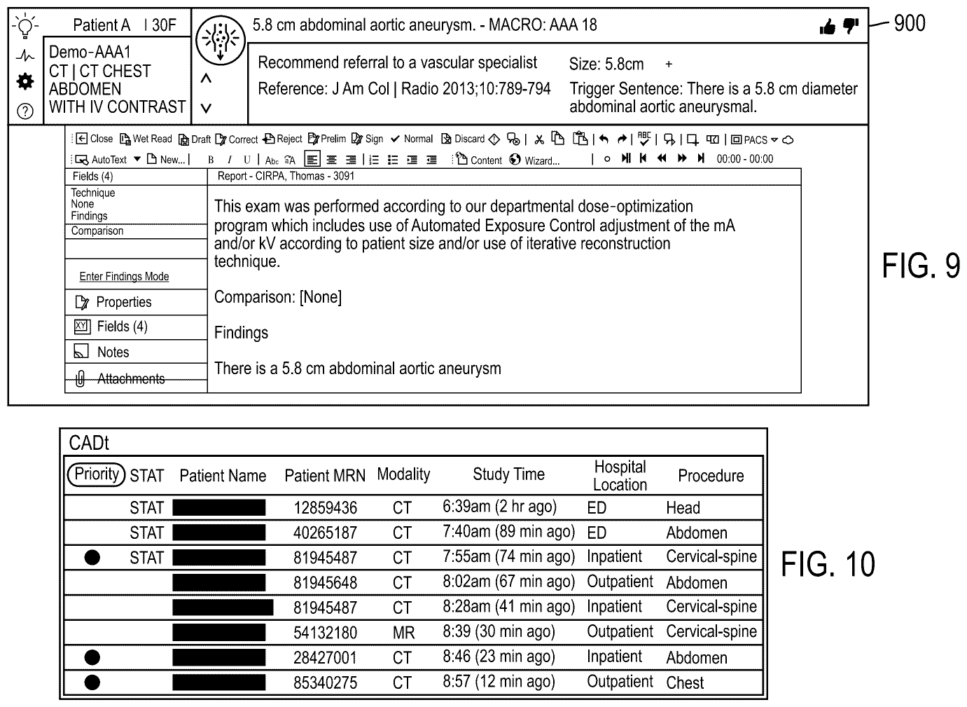

FIG. 8 shows a user interface 800 of the recoMD® program that color codes the patient information that appears when the examination is opened. A first colored screen 802, such as a blue screen, provides patient information and some information about the exam. In this way, the radiologist is reviewing the required information and can see the trigger sentence, which references a 5.8 centimeter abdominal aortic aneurysmal (AAA) 804 in a second colored screen 806. This information was extracted from the radiology report and the recommendation is a referral and a one-year follow-up, and the reference to the article providing the recommendation is also provided. The radiologist may copy the information in FIG. 8 and paste that into the report. The “thumbs up” icon 808 may be clicked to insert the presented information into the radiologist report automatically. Also, by dictating or saying a command, all the information from the interface of FIG. 8 may be inserted into the report, to save the time of the radiologist having to determine and insert the information. The recommendation may come from a reference or article and provide the information on best recommendation to include in the report 900 shown in FIG. 9.

Further, the presented information will be specific not only for the provided pathology but tailored to that specific patient. For instance, according to reference material, there may be a low patient risk or a high patient risk. If the patient has emphysema or a family history of cancer, then they are a high-risk patient and would receive a recommendation specific to a high patient risk. If the AI detects a dictated condition of emphysema, then it is trained to automatically only provide the high-risk recommendation. The AI may generate the impression for the radiologist report based on a summarization of the radiologist findings and how the radiologist dictates information.

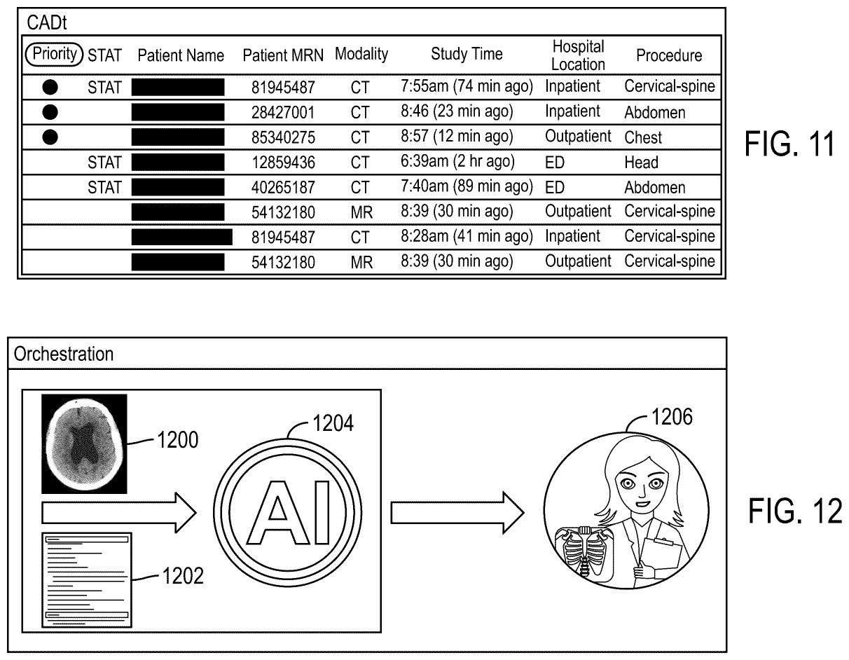

FIG. 10 shows the output from a CAD (computer aided detection) triage (CADt) model to include in the worklist. The CADt is a detection model, shown as 702 in FIG. 7. If CADt detects a certain kind of critical finding, then the CADt should mark the study as critical on the list of FIG. 10. FIG. 11 shows how the results may be sorted by critical positive studies appearing at the top. The CADt model allows the radiologist to read an exam faster that has a positive finding.

For CADt, positive cases with a diagnosis are only found in a minority of studies. For instance, the incidence of intracranial hemorrhage occurs about 5% of non-contrast head CT exams and pulmonary emboli are seen about 7% of the time. The vast majority of studies are negative. If the CADt has a high negative predictive value or high confidence level for negative findings, then the radiologist can more efficiently read the exams marked as negative by the CADt algorithm. In addition, AI has been known to find pathologies that human observers miss. The algorithm may identify features and conditions the user may miss, such as very subtle intracranial hemorrhage along the falx or the calvarium.

AI can also be used to provide more learning opportunities through an AI augmented peer learning system. The error rate in chest x-rays ranges from 10% to 30% and can be as high as 40% in some cases. For instance, if the error rate is 20% and there are 190 chest x-rays, 38 chest x-rays will have errors. If the sensitivity of the AI algorithm for detecting pathology is 95%, AI will identify 36 of the 38 errors, whereas a typical manual peer review process would detect only 1-2 errors. The AI only currently reviews a limited set of conditions, but the conditions are increasing continually.

Orchestration, also referred to as a context driven workflow manager, controls the movement of data to an AI program and the movement of data to destinations after AI processing. Information is extracted from an image 1200 or report 1202 and sent to an AI program 1204 as shown in FIG. 12. The AI may then extract the information from the image, such as no intracranial hemorrhage, and passes the information downstream to the radiologist 1206. The arrows are the data movement, which is the orchestration.

Orchestration is required to send the study to an appropriate AI algorithm. If the study is appropriate for intracranial hemorrhage evaluation (e.g., the study is a non-contrast head CT, and the series chosen is the axial soft tissue kernel), then the study will be sent to an AI algorithm for intracranial hemorrhage detection. When the right data is sent to the right AI algorithm, the AI algorithm will interpret the data and provide a structured result as output. Orchestration is required to ensure the right images (or series of images) is sent to the right AI algorithm. A poor quality orchestration system will result in poor quality AI outputs.

The orchestration not only needs to evaluate the images to select the appropriate image orientation matching the data upon which the AI was trained, but high quality orchestration systems can also review the patient data, study protocol and other parameters to ensure it will produce a high quality result. For instance, if a study had suboptimal contrast opacification of the relevant anatomy, a high quality orchestrator can send the study to an AI algorithm that was trained on and hence optimized for interpreting exams with suboptimal contrast timing. In summary, a high quality orchestration system was ensure the right series of the right study for the right patient is sent to the right AI algorithm.

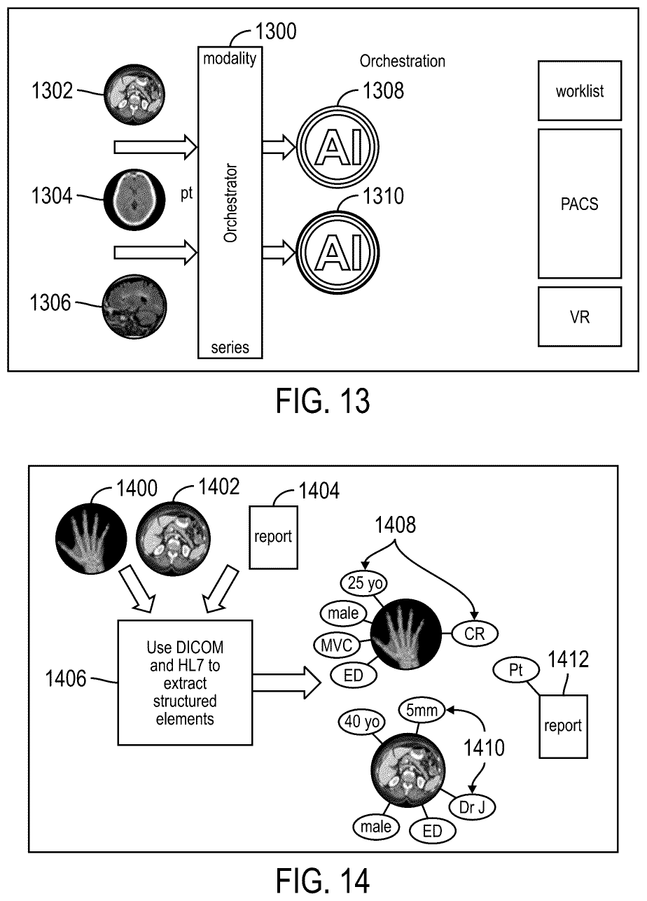

FIG. 13 shows how the orchestrator engine 1300 is located between the image data 1302, 1304, 1306 and the AI models 1308, 1310. The orchestrator must understand the different components of the data to determine the AI program most suited to process the data and the modality of the source of the data.

The orchestrator needs to know the content of the data. The orchestrator may not be able to determine how to forward the data if all the data, including images and patient data, is unstructured.

FIG. 14 illustrates the situation where there are unstructured images 1400, 1402 and a report 1404. Data standards 1406 add structure to the data, such as through DICOM and HL7, both of which provide some structured information 1408, 1410, 1412 about the content of the examination and the report. However that data is limited, not always accurate, and requires human intervention to validate the output. For instance, the HL7 order may indicate the study should be performed with intravenous contrast, however when the technician performed the actual study for several different reasons, the exam may have been performed without intravenous contrast. In this case, the HL7 metadata may not correctly describe the content of the image.

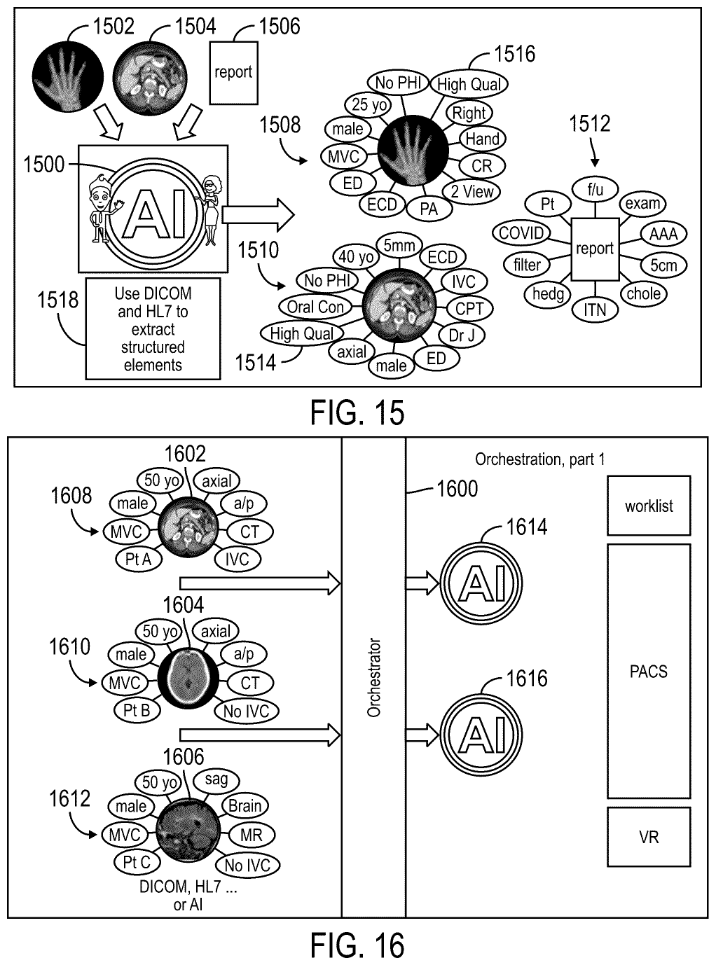

FIG. 15 shows how AI 1500 may be used to process the images 1502, 1504 and report 1506 and extract information, such as correctly interpret the content of the image, e.g., identify an image is an x-ray of a right hand and the correctly identify each of the x-ray views. The AI 1500 uses computer vision to process the image and can standardize the labels and metadata 1508, 1510, 1512 to add more accurate and more robust structure to the unstructured radiology data. The AI 1500 can provide information not included in the DICOM and HL7 data, such as indicate if the exam is high quality 1514,1516. AI 1500 can also process the radiology reports 1506 using NLP to extract information to include in metadata 1508, 1510, 1512 in a normalized fashion, so all the metadata and descriptive data in the report is in a standardized format. In this way, the AI 1500 provides an improved way to structure information from unstructured data and is more accurate, robust and requires less human interaction than current techniques of simply accessing metadata from DICOM and HL7 1518.

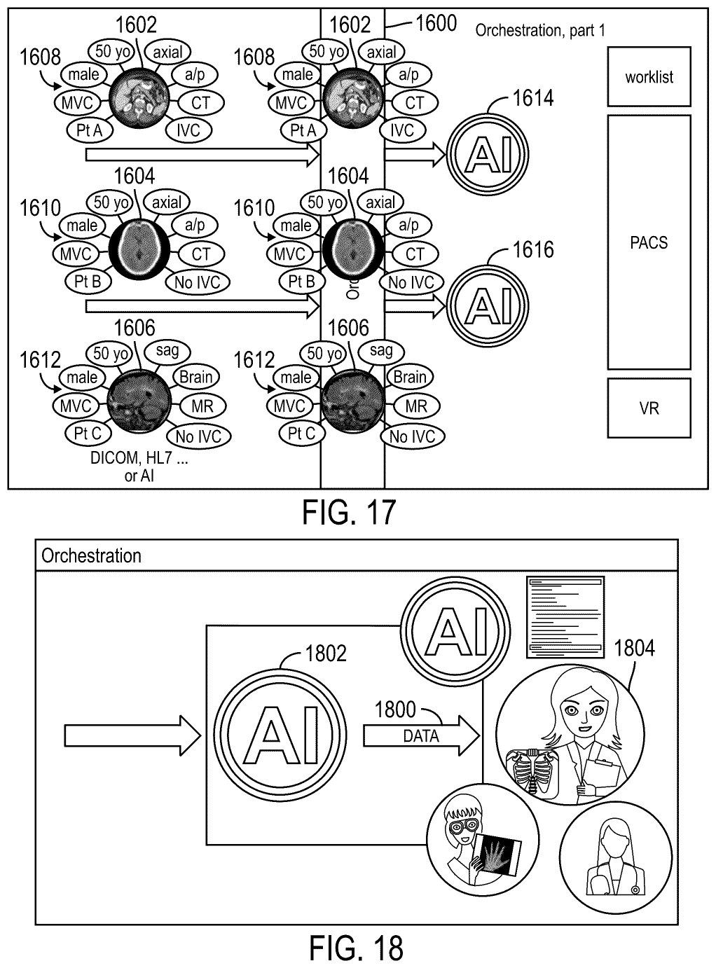

FIG. 16 shows how the orchestrator 1600 receives the images 1602, 1604, 1606 supplemented with the structured information (labels and metadata) 1608, 1610, 1612 from DICOM, HL7 or AI. With improved labeling and metadata 1608, 1610, 1612 from the AI, the orchestrator engine 1600 can make better decisions about how to further process and forward the data, also as shown in FIG. 17, and can better determine the most appropriate AI model 1614, 1616 to forward the data for further processing. The orchestrator 1600 also uses this structured information to determine what data is not appropriate to send to any AI model 1614, 1616 and can thereby bypass this step and instead send the information downstream. The oval bubbles 1608, 1610, 1612 surrounding the images 1602, 1604, 1606 depict the structured data that is used by the orchestrator engine 1600 to make the determinations for the most appropriate location for the data to be sent.

Optimal data orchestration allows for automated data movement that is informed by the content of the data, i.e., an automated, context driven workflow. As shown in FIG. 18, the structured output 1800 from the AI model 1802 (e.g., positive or negative intracranial hemorrhage) is sent by the AI 1802 vendor to the radiologist 1804 (usually through the PACS or through an AI user interface that interacts with the PACS). However, if an orchestrator intercepts the structured AI output, the orchestration has far more options in which it can forward the data, such as to different AI solutions, to a radiology technologist, to a radiology assistant, etc., before or concurrently with the data being sent to the radiologist. For instance, if a CT image of the spine is received but is missing required reconstructions for optimal interpretation, then instead of sending the study to the radiologists, the orchestrator may send the CT image back to the radiology technician to produce the necessary reconstructions. This process prevents incomplete studies from being sent to the radiologist worklist and avoids both delaying exam interpretation along with unnecessarily using radiologist time for administrative tasks.

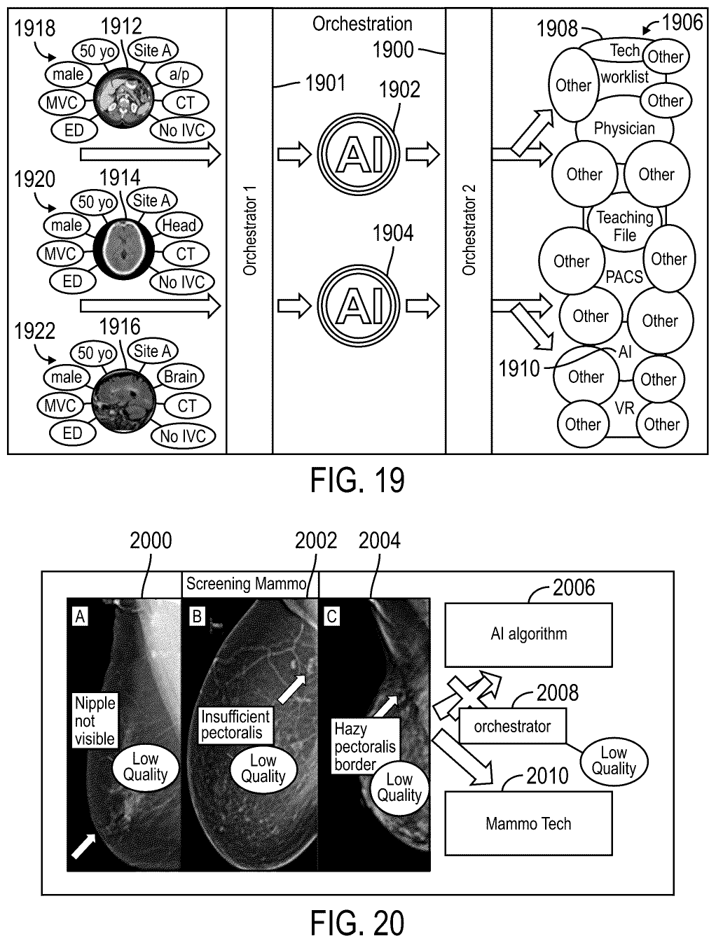

FIG. 19 shows how a second orchestrator 1900 can further process reports and messages from the AI 1902, 1904 to determine whether the report/image should be forwarded to other destinations 1906 before or in addition to reaching the physician, such as the radiologist technician 1908, teaching file, further AI processing 1910, etc. The second orchestrator 1900 provides post-processing after orchestrator 1901 determines the AI 1902, 1904 to process the images 1912, 1914, 1916 and their metadata 1918, 1920, 1922.

FIG. 20 shows several examples of poor quality mammogram images 2000, 2002, 2004. An AI model 2006 can be implemented to determine the quality of mammographic images. The results of this model 2006 can be presented to the data orchestrator 2008, which gives the orchestrator 2008 to ability to provide different options for data movement. For instance, the orchestrator 2008 can be programmed to make a more intelligent choice for what to do with the poor-quality image. For instance, instead of sending the image to an AI system, the orchestrator 2008 could instead send the image back to the radiology technician 2010 to improve the image quality, and/or forward the low-quality study to the radiologist with a message noting the suboptimal image quality and forward this information for automated inclusion into the radiology report, and/or create a list of suboptimal image quality for future quality control. All these additional annotations assist the orchestrator 2008 in determining what to do with the exam. The orchestrator can manage the workflow in real time to ensure data is going to most optimal target, such as further AI processing, back to the technician, forwarded to the radiologist with or without information on processing results. The orchestrator 2008 may further decide no further processing is needed and the data can go to the work list and PACS to reach the radiologist.

As shown in FIG. 19, the orchestrator 1901, 1900 may determine where to forward the images/reports 1912, 1914, 1916 before processing by the AI engine 1902, 1904 and after processing by the AI engine, a second level orchestration 1900. This provides the context driven workflow to use the context of the content of the information to determine the workflow and add further structure to assist in decision making.

However, there may be some vendors that the image/report should not be sent and there will be instances in which the exam is reported by the radiologist before processed by the relevant AI system. The orchestrator will need to know of such communication limitations and the timing of every data relevant event so that it can react appropriately. The orchestrator engine is not only placed between the data and the AI system, but also placed between the AI systems and clinical applications.

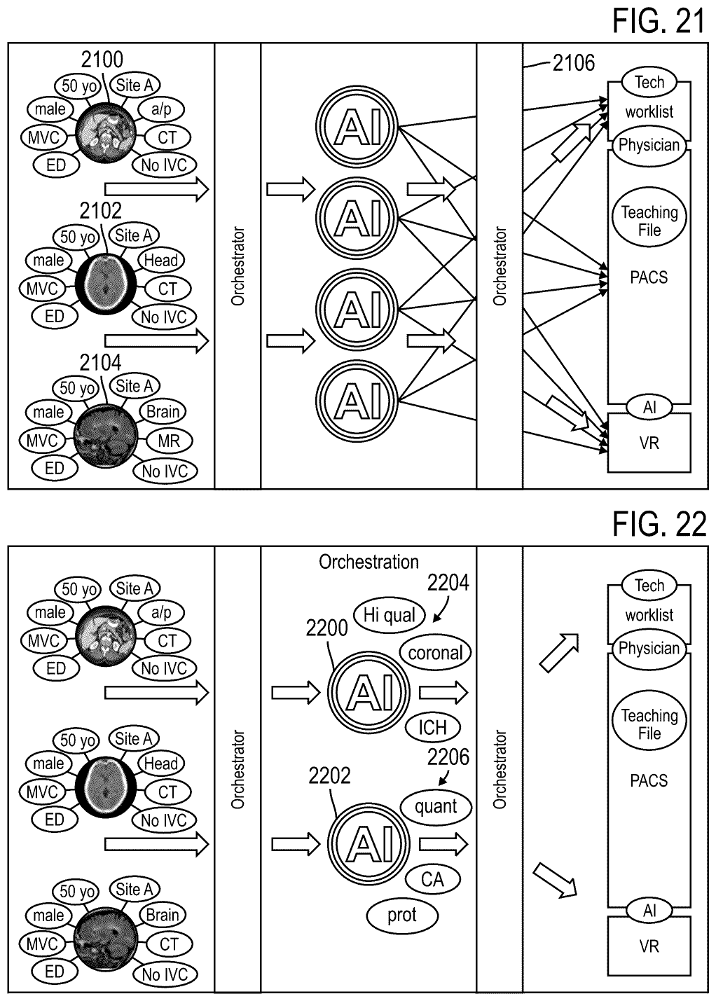

FIG. 21 shows how a report/image 2100, 2102, 2104 may flow to different destinations and vendors, and the orchestrator 2106 has control over how data flows to the downstream systems.

AI models may only interpret a component of any study (e.g., a particular series or a single image). The orchestrator engine manages this limitation by using the structured information about the images and series of each exam to determine the appropriate next destination. Once the appropriate component of an exam is interpreted by the AI model, the orchestrator engine also captures that result and uses that additional structure for downstream data movement. This is not how current AI systems are implemented. Without an orchestrator engine collecting the data from the AI system, that data is lost and cannot be used for automated, intelligent subsequent data movement (i.e., context driven workflow).

For this reason, it is important is to add the structured information provided by the AI model to the original data so that an orchestrator engine can use that result to automate intelligent workflows. FIG. 22 shows how the orchestrator AI engine 2200, 2202 adds structured labels 2204 and 2206 to the data, and how data that normally discarded in current AI implementations can be collected and utilized for subsequent intelligent workflow automation.

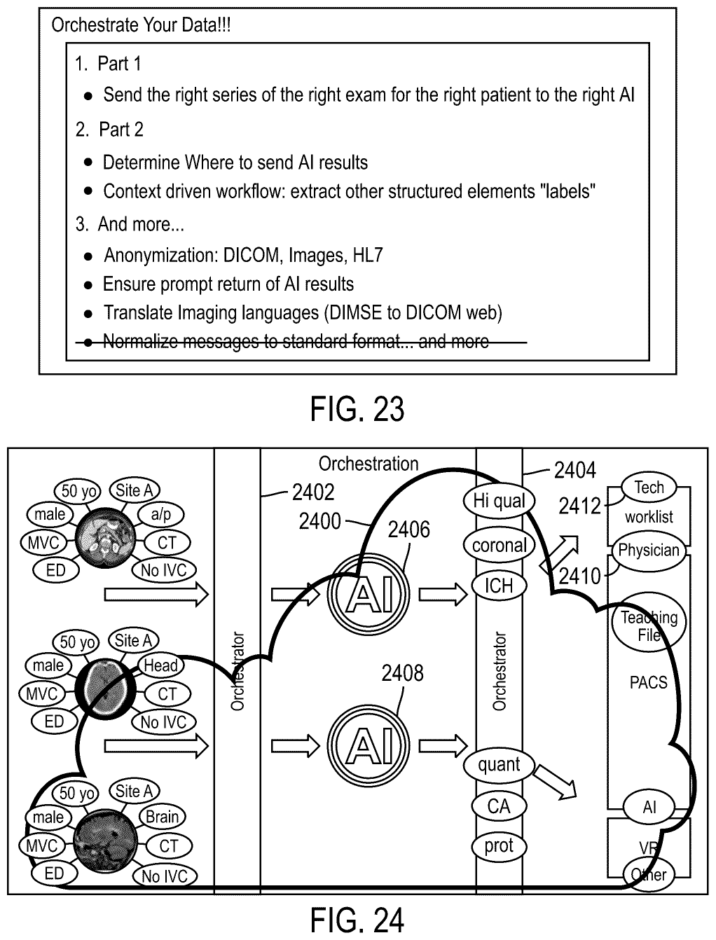

FIG. 23 illustrates the flow of the orchestration of the data to provide the right series of the right exam for the right patient to the right AI engine. In this way, the data is not just sent to the PACS for delivery to the radiologist, but the orchestrator may send for other processing, such as by further AI engines, other applications, or other data collection systems. The orchestrator engine may also handle translating imaging languages, e.g., DIMSE to DICOM web, normalizing DICOM and HL7 data, tracking and logging data movement, and anonymizing data.

FIG. 24 shows how the orchestration process to add value to the data is managed in the cloud. The data may be sent to the cloud 2400 for orchestration 2402, 2404 and AI processing 2406, 2408 before being forwarded to the physician 2410, tech 2412, or on premises clinical application. The cloud-based system 2400 may include cloud native services, such as Kubernetes, to optimize the capabilities of the orchestration and implement the AI engine as cloud microservices or through cloud-based APIs.

The radiologists will receive the data processed by the orchestrator and AI models and that additional information will aid the radiologist in their image interpretation and workflow. This allows the radiologist to understand how the AI models process the information and how data orchestration works so that the radiologist can understand the output of these systems.

In described embodiments, the orchestrator automates context driven workflows, by capturing the results from AI models and other software to add structure to unstructured radiology data. Orchestration is also an essential component to improve the output of any AI model by ensuring the most appropriate image or series of the right study for the right patient is provided to the right AI system.

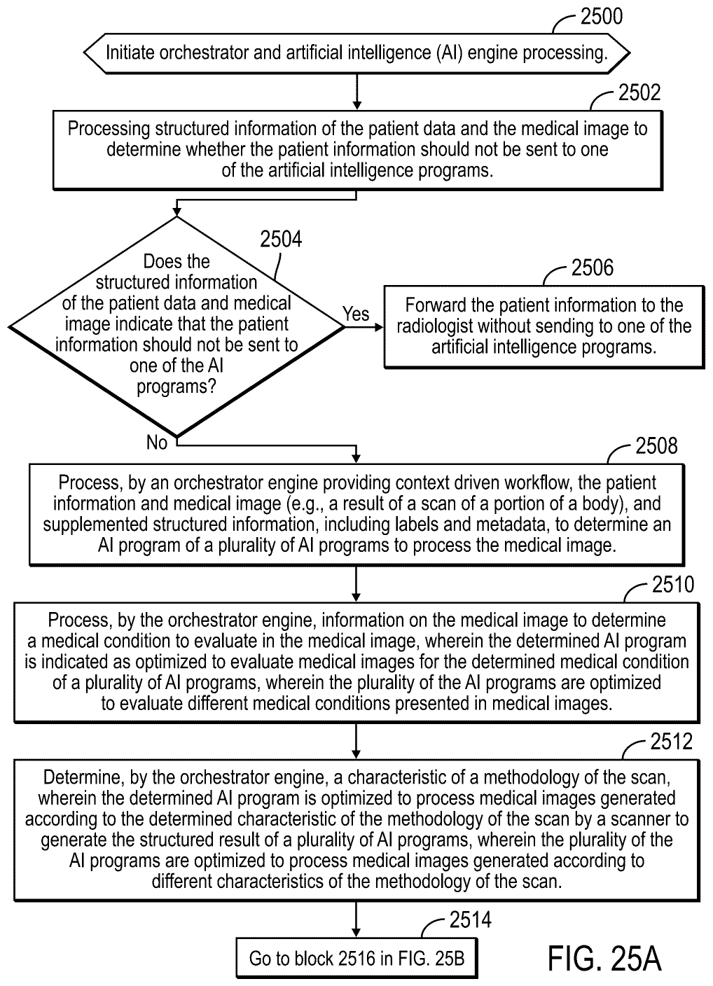

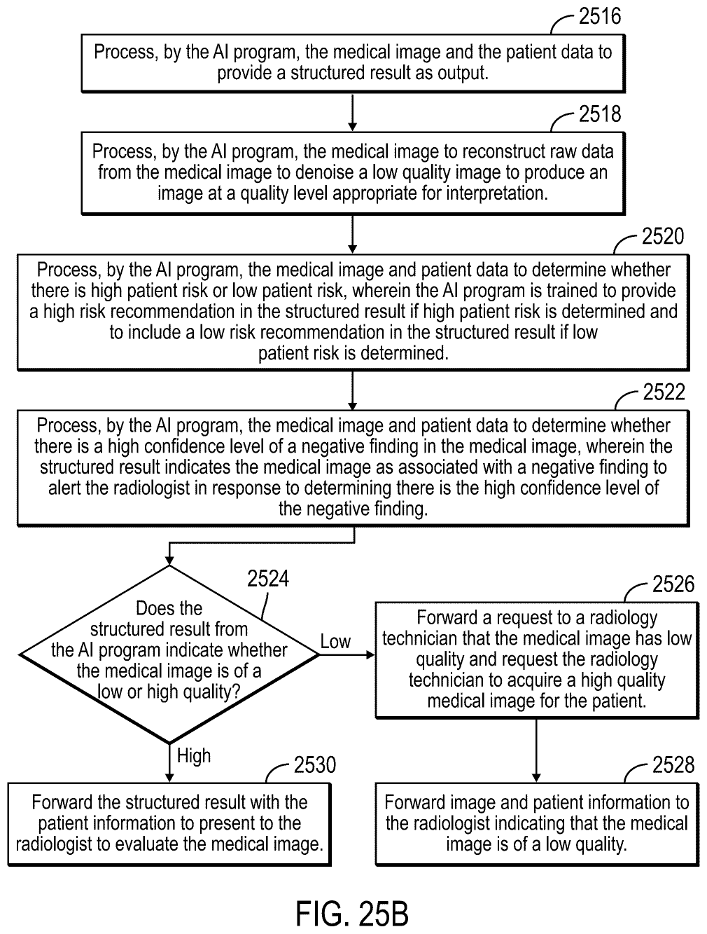

FIGS. 25A and 25B illustrate an embodiment of operations performed by the orchestrator and AI engine, such as these components shown in FIGS. 13, 15-19, 21, 22, and 24, described above. Upon initiating (at block 2500) orchestrator and AI engine processing, an orchestrator engine, providing a context driven workflow, processes (at block 2502) structured information of the patient information and medical image (e.g., a result of a scan of a portion of a body) to determine whether the patient information should be sent to one of the AI programs. If (at block 2504) the structured information of the patient data and medical image indicate that the patient information should not be sent to one of the AI programs, then the orchestrator forwards (at block 2506) the patient information to the radiologist without sending to one of the artificial intelligence programs.

If (at block 2504) the structured information of the patient data and medical image indicate that the patient information should be sent to one of the AI programs, then the orchestrator engine, providing context driven workflow, processes (at block 2508) the patient information and medical image (e.g., a result of a scan of a portion of a body), and supplemented structured information, including labels and metadata, to determine an AI program of a plurality of AI programs to process the medical image. In addition, the orchestrator engine may perform the operations at blocks 2510 and/or 2512. At block 2510, the orchestrator engine processes (at block 2510) information on the medical image to determine a medical condition to evaluate in the medical image. The determined AI program is indicated as optimized to evaluate medical images for the determined medical condition of a plurality of AI programs. The plurality of the AI programs may be optimized to evaluate different medical conditions presented in medical images. At block 2512, the orchestrator engine determines a characteristic of a methodology of the scan. The determined AI program is optimized to process medical images generated according to the determined characteristic of the methodology of the scan by a scanner to generate the structured result of a plurality of AI programs. The plurality of the AI programs are optimized to process medical images generated according to different characteristics of the methodology of the scan. The different characteristics of the methodology of the scan performed by a scanner may concern at least one of characteristics of software used, scanner types, versions of the scanner, quality of the scan, contrast timing of the scan, and scanning protocols.

At block 2514, control proceeds to block 2516 in FIG. 25B to proceed to processing by the AI program. At block 2516, the AI program processes the medical image and the patient data to provide a structured result as output. In certain embodiments, the AI program may use computer vision to determine a body part shown in the medical image and the structured result includes information on the body part shown in the medical image. Additionally and optionally, the AI program may perform the operations at blocks 2518, 2520, 2522, 2524, 2526, and 2528. The AI program may process (at block 2518) the medical image to reconstruct raw data from the medical image to denoise a low quality image to produce an image at a quality level appropriate for interpretation. The AI program may further process (at block 2520) the medical image and patient data to determine whether there is high patient risk or low patient risk. The AI program may be trained to provide a high risk recommendation in the structured result if high patient risk is determined and to include a low risk recommendation in the structured result if low patient risk is determined. The AI program may further be trained to process (at block 2522) the medical image and patient data to determine whether there is a high confidence level of a negative finding in the medical image. The structured result indicates the medical image as associated with a negative finding to alert the radiologist in response to determining there is the high confidence level of the negative finding.

A determination is made (at block 2524) whether the structured result from the AI program indicates whether the medical image is of a low or high quality. If (at block 2524) the quality is low, then the orchestrator engine forwards (at block 2526) a request to a radiology technician that the medical image has low quality and request the radiology technician to acquire a high quality medical image for the patient. The orchestrator engine may further forward (at block 2528) the image and patient information to the radiologist indicating that the medical image is of a low quality. If (at block 2524) the image is of high quality, then the structured result may be forwarded (at block 2530) with the patient information to present to the radiologist to evaluate the medical image.

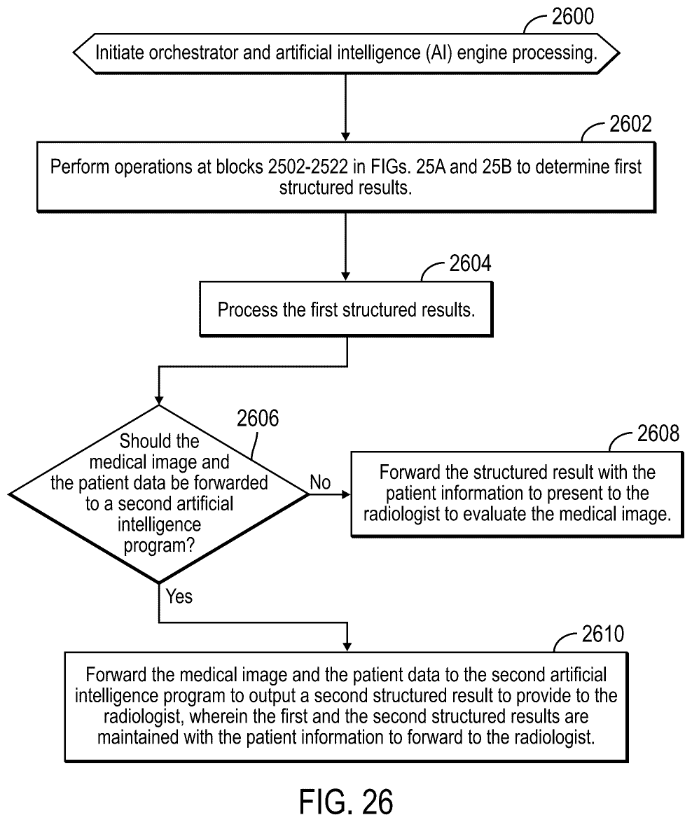

FIG. 26 illustrates an embodiment of operations performed by the orchestrator and AI engine, such as these components shown in FIGS. 13, 15-19, 21, 22, and 24, described above. Upon initiating (at block 2600) orchestrator and AI engine processing, the operations at blocks 2502-2522 in FIGS. 25A and 25B are performed (at block 2602) to output first structured results. The first structured output results are processed (at block 2604) and a determination is made (at block 2606) whether the medical image and the patient data should be forwarded to a second AI program. If not, then the structured results with the patient information are forwarded (at block 2610) to present to the radiologist to evaluate the medical image. If (at block 2606) the medical image and patient data should be forwarded, then the orchestration engine forwards (at block 2610) the medical image and the patient data to the second artificial intelligence program to output a second structured result second structured result to provide to the radiologist. The first and the second structured results are maintained with the patient information to forward to the radiologist.

The program components used to implement the AI, NLP, orchestrator and other components in FIG. 7, 12-19, 21-24 may comprise routines, programs, objects, components, logic, data structures, and so on that perform particular tasks or implement particular abstract data types. The program components FIG. 7, 12-19, 21-24, including the orchestrators and AI engine s, may be implemented in in one or more computer systems, where if they are implemented in multiple computer systems, then the computer systems may communicate over a network.

The program components in FIG. 7, 12-19, 21-24 may be accessed by a processor from memory to execute. Alternatively, some or all of the program components in FIG. 7, 12-19, 21-24 may be implemented in separate hardware devices, such as Application Specific Integrated Circuit (ASIC) hardware devices.

The functions described as performed by the program components of FIG. 7, 12-19, 21-24 may be implemented as program code in fewer program modules than shown or implemented as program code throughout a greater number of program modules than shown.

The described program components, including, but not limited to, the orchestration program, the AI programs, and other program components in FIG. 7, 12-19, 21-24, may use machine learning and deep learning algorithms, such as decision tree learning, association rule learning, neural network, inductive programming logic, support vector machines, Bayesian network, etc. For artificial neural network program implementations, the neural network may be trained using backward propagation to adjust weights and biases at nodes in a hidden layer to produce the classification based on patient information, including a medical image and patient data. In backward propagation used to train a neural network machine learning module, biases at nodes in the hidden layer are adjusted accordingly to produce the information having specified confidence levels the based on a patient medical image and patient data. Backward propagation may comprise an algorithm for supervised learning of artificial neural networks using gradient descent. Given an artificial neural network and an error function, the method may calculate the gradient of the error function with respect to the neural network's weights and biases.

In one embodiment, the orchestrator and AI programs may comprise a machine learning program that is trained using a training set comprising sets of patient data, including medical images and patient data, that have been classified with a ground truth classification, and the AI programs are trained to produce the ground truth classifications provided for the training set of reports. The AI program and orchestrator would then be trained with those findings to produce the output assigned to those findings and observations. The orchestrator would use the output of any AI programs and other technical solutions that provide structure from the unstructured radiology data to automate concurrent and downstream workflows.

In an alternative embodiment, the program components of FIG. 7, 12-19, 21-24 may be implemented not as a machine learning module, but implemented using a rules based system to determine the outputs from the inputs. The program components may further be implemented using an unsupervised machine learning module, or machine learning implemented in methods other than neural networks, such as multivariable linear regression models.

The present invention may be a system, a method, and/or a computer program product. The computer program product may include a computer readable storage medium (or media) having computer readable program instructions thereon for causing a processor to carry out aspects of the present invention.

The computer program product comprises a computer readable storage medium implemented using standard programming and/or engineering techniques to produce software, firmware, hardware, or any combination thereof. The described operations may be implemented as code or logic maintained in a “computer readable storage medium”. The term “code” and “program code” as used herein refers to software program code, hardware logic, firmware, microcode, etc. The computer readable storage medium, as that term is used herein, includes a tangible element, including at least one of electronic circuitry, storage materials, a casing, a housing, a coating, hardware, and other suitable materials. A computer readable storage medium may comprise, but is not limited to, a magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), Solid State Devices (SSD), computer encoded and readable punch cards, etc. The computer readable storage medium may further comprise a hardware device implementing firmware, microcode, etc., such as in an integrated circuit chip, a programmable logic device, a Programmable Gate Array (PGA), field-programmable gate array (FPGA), Application Specific Integrated Circuit (ASIC), etc. A computer readable storage medium is not comprised solely of transmission signals and includes physical and tangible components. Those skilled in the art will recognize that many modifications may be made to this configuration without departing from the scope of the present invention, and that the article of manufacture may comprise suitable information bearing medium known in the art.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

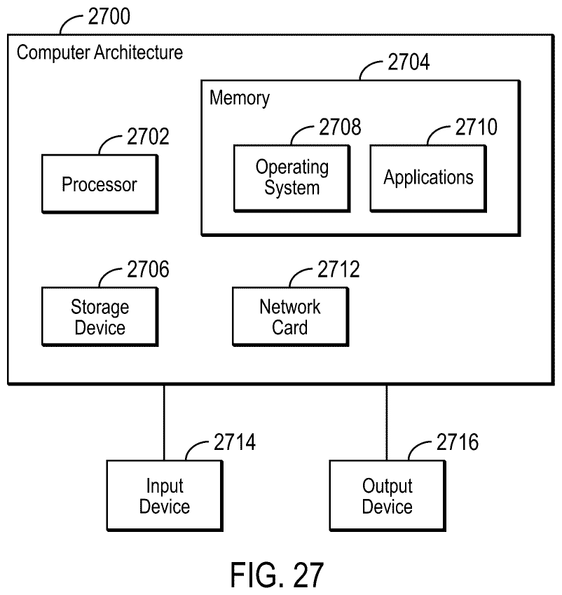

The program components of FIG. 7, 12-19, 21-24 may be implemented in one or more computer systems, having a computer architecture 2700 as shown in FIG. 27, and including a processor 2702 (e.g., one or more microprocessors and cores), a memory 2704 (e.g., a volatile memory device), and storage 2706 (e.g., a non-volatile storage, such as magnetic disk drives, solid state devices (SSDs), optical disk drives, a tape drive, etc.). The storage 2706 may comprise an internal storage device or an attached or network accessible storage. Programs, including an operating system 2708 and applications 2710 stored in the storage 2706 are loaded into the memory 2704 and executed by the processor 2702. The applications 2710 may include the orchestrator engine, AI programs and other program components of FIG. 7, 12-19, 21-24. The architecture 2700 further includes a network card 2712 to enable communication with a network. An input device 2714 is used to provide user input to the processor 2702, and may include a keyboard, mouse, pen-stylus, microphone, touch sensitive display screen, or any other activation or input mechanism known in the art. An output device 2716, such as a display monitor, printer, storage, etc., is capable of rendering information transmitted from a graphics card or other component. The output device 2716 may render the GUIs described with respect to figures and the input device 2714 may be used to interact with the graphical controls and elements in the GUIs described above. The architecture 2700 may be implemented in any number of computing devices, such as a server, mainframe, desktop computer, laptop computer, hand held computer, tablet computer, personal digital assistant (PDA), telephony device, cell phone, etc.

The terms “an embodiment”, “embodiment”, “embodiments”, “the embodiment”, “the embodiments”, “one or more embodiments”, “some embodiments”, and “one embodiment” mean “one or more (but not all) embodiments of the present invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the present invention need not include the device itself.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims herein after appended.

1. A computer program product for processing patient information, including a medical image for a patient and patient data, to provide to a radiologist, the computer program product comprising a computer readable storage medium having computer readable program code embodied therein that is executable to perform operations, the operations comprising:
processing, by an orchestrator engine providing context driven workflow, the patient information to determine an artificial intelligence program of a plurality of artificial intelligence programs to process the medical image;
processing, by the artificial intelligence program, the medical image and the patient data to provide a structured result as output; and
forwarding the structured result with the patient information to the radiologist to evaluate the medical image.

2. The computer program product of claim 1, wherein the medical image is a result of a scan of a portion of a body of the patient, wherein the processing the patient information to determine the artificial intelligence program further comprises:
processing information on the medical image to determine a medical condition to evaluate in the medical image, wherein the determined artificial intelligence program is indicated as optimized to evaluate medical images for the determined medical condition of a plurality of artificial intelligence programs, wherein the plurality of the artificial intelligence programs are optimized to evaluate different medical conditions presented in medical images.

3. The computer program product of claim 1, wherein the medical image is a result of a scan of a portion of a body of the patient, wherein the processing the patient information to determine the artificial intelligence program further comprises:
determining a characteristic of a methodology of the scan, wherein the determined artificial intelligence program is optimized to process medical images generated according to the determined characteristic of the methodology of the scan by a scanner to generate the structured result of a plurality of artificial intelligence programs, wherein the plurality of the artificial intelligence programs are optimized to process medical images generated according to different characteristics of the methodology of the scan.

4. The computer program product of claim 3, wherein the different characteristics of the methodology of the scan performed by a scanner concern at least one of characteristics of software used, scanner types, versions of the scanner, quality of the scan, contrast timing of the scan, and scanning protocols.

5. The computer program product of claim 1, wherein the artificial intelligence program processes the medical image to reconstruct raw data from the medical image to denoise a low quality image to produce an image at a quality level appropriate for interpretation.

6. The computer program product of claim 1, wherein the artificial intelligence program processes the medical image and patient data to determine whether there is high patient risk or low patient risk, wherein the artificial intelligence program is trained to provide a high risk recommendation in the structured result if high patient risk is determined and to include a low risk recommendation in the structured result if low patient risk is determined.

7. The computer program product of claim 1, wherein the artificial intelligence program processes the medical image and patient data to determine whether there is a high confidence level of a negative finding in the medical image, wherein the structured result indicates the medical image as associated with a negative finding to alert the radiologist in response to determining there is the high confidence level of the negative finding.

8. The computer program product of claim 1, wherein the artificial intelligence program uses computer vision to determine a body part shown in the medical image, wherein the structured result includes information on the body part shown in the medical image.

9. The computer program product of claim 1, wherein the patient data and medical image are supplemented with structured information, including labels and metadata, and wherein the orchestrator engine processes the structured information to determine the artificial intelligence program.

10. The computer program product of claim 1, wherein the patient data and medical image are supplemented with structured information, including labels and metadata, wherein the orchestrator engine further performs:
processing the structured information of the patient data and the medical image to determine whether the patient information should not be sent to one of the artificial intelligence programs; and
forwarding the patient information to the radiologist without sending to one of the artificial intelligence programs in response to determining that the patient information should not be sent to one of the artificial intelligence programs.

11. The computer program product of claim 1, the structured result from the artificial intelligence program indicates whether the medical image is of a low quality or a high quality, wherein the orchestrator engine further performs:
determining whether the structured result indicates the medical image is of a low quality; and
in response to determining that the medical image is of the low quality, forwarding a request to a radiology technician that the medical image has low quality and requesting the radiology technician to acquire a high quality medical image for the patient.

12. The computer program product of claim 1, the structured result from the artificial intelligence program indicates whether the medical image is of a low quality or a high quality, wherein the orchestrator engine further performs:
determining whether the structured result indicates the medical image is of a low quality; and
in response to determining that the medical image is of the low quality, indicating to the radiologist receiving the patient information that the medical image is of a low quality.

13. The computer program product of claim 1, wherein the artificial intelligence program comprises a first artificial intelligence program, wherein the structured result comprises a first structured result, wherein the orchestrator engine further performs:
processing the first structured result to determine to forward the medical image and the patient data to a second artificial intelligence program; and
forwarding the medical image and the patient data to the second artificial intelligence program to output a second structured result to provide to the radiologist, wherein the first and the second structured results are maintained with the patient information to forward to the radiologist.

14. A system for processing patient information, including a medical image for a patient and patient data, to provide to a radiologist, comprising:
a plurality of artificial intelligence programs to process a medical image;
an orchestrator engine, providing a context driven workflow, to process the patient information to determine an artificial intelligence program of a plurality of artificial intelligence programs to process the medical image;
wherein the artificial intelligence program processes the medical image and the patient data to provide a structured result as output; and
wherein the orchestration engine forwards the structured result with the patient information to the radiologist to evaluate the medical image.

15. The system of claim 14, wherein the medical image is a result of a scan of a portion of a body of the patient, wherein the processing the patient information to determine the artificial intelligence program further comprises:
processing information on the medical image to determine a medical condition to evaluate in the medical image, wherein the determined artificial intelligence program is indicated as optimized to evaluate medical images for the determined medical condition of a plurality of artificial intelligence programs, wherein the plurality of the artificial intelligence programs are optimized to evaluate different medical conditions presented in medical images.

16. The system of claim 14, wherein the medical image is a result of a scan of a portion of a body of the patient, wherein the processing the patient information to determine the artificial intelligence program further comprises:
determining a characteristic of a methodology of the scan, wherein the determined artificial intelligence program is optimized to process medical images generated according to the determined characteristic of the methodology of the scan by a scanner to generate the structured result of a plurality of artificial intelligence programs, wherein the plurality of the artificial intelligence programs are optimized to process medical images generated according to different characteristics of the methodology of the scan.

17. The system of claim 14, wherein the patient data and medical image are supplemented with structured information, including labels and metadata, wherein the orchestrator engine further performs:
processing the structured information of the patient data and the medical image to determine whether the patient information should not be sent to one of the artificial intelligence programs; and
forwarding the patient information to the radiologist without sending to one of the artificial intelligence programs in response to determining that the patient information should not be sent to one of the artificial intelligence programs.

18. A computer implemented method for processing patient information, including a medical image for a patient and patient data, to provide to a radiologist, comprising:
processing, by an orchestrator engine providing context driven workflow, the patient information to determine an artificial intelligence program of a plurality of artificial intelligence programs to process the medical image;
processing, by the artificial intelligence program, the medical image and the patient data to provide a structured result as output; and
forwarding the structured result with the patient information to the radiologist to evaluate the medical image.

19. The computer implemented method of claim 18, wherein the medical image is a result of a scan of a portion of a body of the patient, wherein the processing the patient information to determine the artificial intelligence program further comprises:
processing information on the medical image to determine a medical condition to evaluate in the medical image, wherein the determined artificial intelligence program is indicated as optimized to evaluate medical images for the determined medical condition of a plurality of artificial intelligence programs, wherein the plurality of the artificial intelligence programs are optimized to evaluate different medical conditions presented in medical images.

20. The computer implemented method of claim 18, wherein the medical image is a result of a scan of a portion of a body of the patient, wherein the processing the patient information to determine the artificial intelligence program further comprises:
determining a characteristic of a methodology of the scan, wherein the determined artificial intelligence program is optimized to process medical images generated according to the determined characteristic of the methodology of the scan by a scanner to generate the structured result of a plurality of artificial intelligence programs, wherein the plurality of the artificial intelligence programs are optimized to process medical images generated according to different characteristics of the methodology of the scan.
